(19) United States
(12) Patent Application Publication
(10) Pub. No.: US 2002/0016317 A1
(43) Pub. Date: Feb. 7, 2002

(54) Sterol ester compositions

(76) Inventors: David Allen Schul, Maineville, OH (US); Roger Stephen Berger, Fairfield, OH (US); John Keeney Howie, Oregonia, OH (US); Eugene Harold Lessen JR., Hamilton, OH (US); Vincent York-Leung Wong, Hamilton, OH (US); Michael Herbert Brock, Loveland, OH (US)

Correspondence Address: THE PROCTER & GAMBLE COMPANY, PATENT DIVISION, IVORYDALE TECHNICAL CENTER - BOX 474, 5299 SPRING GROVE AVENUE, CINCINNATI, OH, 45217, US

(21) Appl. No.: 09/812,532

(22) Filed: Mar. 20, 2001

Related U.S. Application Data
(60) Provisional application No. 60/192,412, filed on Mar. 27, 2000.

(51) Int. Cl. A61K 31/56; A23D 9/007

(52) U.S. Cl. 514/182; 426/601

(57) ABSTRACT

Sterol ester compositions having fatty acid moieties comprising greater than 50% monounsaturated fatty acids (MUFAs), preferably from about 55% to about 80% MUFAs, and more preferably from about 60% to about 75% MUFAs. Preferably, the fatty acid moieties comprise less than about 6% saturated fatty acids (SFAs), more preferably from about 0.1% to about 4% SFAs, and most preferably from about 0.5% to about 2% SFAs. The fatty acid moieties of the sterol ester compositions comprise 50% or less polyunsaturated fatty acids (PUFAs). Also disclosed are methods for preparing the sterol ester compositions and the products comprising them.

### CROSS REFERENCE TO RELATED APPLICATION

This application claims the benefit of priority to U.S. Provisional Application Serial No. 60/192,412, filed Mar. 27, 2000, which is herein incorporated by reference.

### TECHNICAL FIELD

The invention relates to sterol ester compositions and their use in edible oils and other food products. Furthermore, the invention relates to methods for preparing the sterol ester compositions and the products comprising them.

### BACKGROUND OF THE INVENTION

A high serum cholesterol level is the most significant single indicator of the risk of cardiovascular disease. It is well accepted that a high cholesterol diet leads to high serum cholesterol levels. Dietary cholesterol absorbed from the gut is introduced to the circulating body plasmas, thus increasing the level of serum cholesterol. Accordingly, lowering the serum cholesterol level is important in preventing and treating cardiovascular disease, especially coronary heart disease.

Because the absorption of dietary cholesterol is of fundamental significance in the regulation of the serum cholesterol level, it is desirable to reduce or prevent the absorption of cholesterol from the gut. Plant sterols are structurally similar to cholesterol and have been known for many years to reduce cholesterol absorption when ingested, thus leading to reduced serum cholesterol levels. Numerous studies show that that plant sterols can effectively be used to lower serum cholesterol levels by reducing the absorption of dietary cholesterol from the gut. See, e.g., Mattson et al., Effect of Plant Sterol Esters on the Absorption of Dietary Cholesterol, J. Nutr. 107:1109-1146 (1977); Pollack, Reduction of Blood Cholesterol in Man, Circulation 7:702-706 (1953); and Grundy et al., The Interaction of Cholesterol Absorption and Cholesterol Synthesis in Man, J. Lipid Res. 10:304 (1969).

Several studies have investigated the hypocholesterolemic properties of sterols and sterols that have been esterified into their fatty acid esters. In one experiment involving rats, edible oils comprising up to 8% sterol esters were shown to reduce the absorption of cholesterol by 20-40%. Mattson et al., Effect of Plant Sterol Esters on the Absorption of Dietary Cholesterol, J. Nutr. 107:1109-1146 (1977). In a study involving humans, test subjects with a high cholesterol diet (500 mg/day) ingested 2 grams of sterol ester dissolved in fat per day. The absorption of cholesterol by the test subjects decreased, on average, by 33%. In the same study, a lower dose (1 gram per day) of free sterols mixed with food decreased cholesterol absorption by 42%. Mattson et al., Optimizing the Effect of Plant Sterols on Cholesterol Absorption in Man, Am. J. Clin. Nutr. 35:697-700 (1982).

Studies of cholesterol metabolism have shown that sterols inhibit the absorption of both endogenic and dietary cholesterol from the intestines. See, e.g., Grundy et al., Effects of Low Dose Phytosterols on Cholesterol Absorption in Man, “Lipoprotein Metabolism,” New York: Springer-Verlag (H. Greten ed., 1976); Kudchodkar et al., Effects of Plant Sterols on Cholesterol Absorption in Man, Atherosclerosis 23:239 (1976). As a result, the excretion of cholesterol in the stool increases, leading to a shortage of cholesterol in the liver and subsequently to a decreased serum cholesterol level. Importantly, sterols have been shown to not affect the absorption of bile acids. See, e.g., Grundy et al., supra.

Although not intending to be limited by theory, it is believed that the hypocholesterolemic function of sterols stems from their ability to displace dietary cholesterol in bile acid micelli. This effect has been observed in animal studies. See, e.g., Ikeda et al., Inhibition of Cholesterol Absorption in Rats by Plant Sterols, J. Lipid Res. 29:1573-1582 (1988); Ikeda et al., Discrimination between Cholesterol and Sitostanol for Absorption in Rats, J. Lipid Res. 29:1583-1591 (1988); Ikeda et al., Effects of Sitosterol and Sitostanol on Micellar Solubility of Cholesterol, J. Nutr. Sci. Vitaminol. 35:361-369 (1989). Similar results have also been observed in humans. See, e.g., Miettinen et al., Bile Salts, Sterols, Sterol Esters, Glycerides and Fatty Acids in Micellar and Oil Phases of Intestinal Contents during Fat Digestion in Man, J. Clin. Chem. Biochem. 9:47-52 (1971).

Plant sterols are natural components of vegetable oils and vegetable fats, and thus are present in small quantities as part of a normal diet. The level found naturally in plants, however, is not sufficient to deliver the desired hypocholesterolemic effects. Accordingly, larger doses must be administered to provide the desired effects. Several ways to introduce plant sterols into the diet have been proposed. For example, U.S. Pat. No. 3,865,939 to Jandacek discloses edible oils comprising from about 2.0 to about 6.9% plant sterols and a solubilizing agent; and U.S. Pat. No. 5,244,887 to Straub discloses a food additive composition comprising a stanol, an edible solubilizing agent, an antioxidant, and a dispersant.

However, plant sterols are exceedingly difficult to solubilize in dietary compositions such as cooking or salad oils. When an efficacious amount of sterols is added to a clear edible oil, the resultant oil has a cloudy appearance and becomes more viscous and “texturized.” Mouthfeel and visual appeal are important factors that consumers consider when evaluating the desirability of a food product. Accordingly, consumers find these viscous, cloudy oils to be unappealing and to have an “unhealthy” appearance despite the health benefits of the products.

Several ways to increase the solubility of the sterols have been suggested. Plant sterol esters are much more soluble in oil than are free (unesterified) sterols, thus making them more useflul in the production of clear edible oils. Accordingly, it has been proposed to use plant sterol esters in oil to lower cholesterol absorption. See, e.g., Mattson et al., Effect of Plant Sterol Esters on the Absorption of Dietary Cholesterol, supra.

U.S. Pat. No. 3,751,569 to Erickson et al. describes cooking and salad oils containing from 0.5% to 10% plant sterol monocarboxylic acid esters. The maximum amount of added plant sterol ester that can be used is that amount which is soluble in the oil at refrigerator temperatures.

U.S. Pat. Nos. 5,502,045 and 5,958,913 to Miettinen et al. describe the use of sitostanol esters and 5α-saturated sterol esters, respectively, in fat based food products for the treatment of hypercholesterolemia.

Although esterification increases sterol solubility, the solubility of the sterol esters taught by the art is still less than desirable for delivering an efficacious quantity of sterols in the form of a clear, non-opaque, appealing edible oil.

In an effort to overcome some of the problems associated with low solubility, PCT published application WO 99/56558 proposes sterol fatty acid esters wherein more than 50% of the fatty acid moieties are polyunsaturated fatty acids (“PUFAs”). The application proposes the use of these compounds in foods and capsules. However, PUFAs have poor oxidative stability, which leads to more rapid degradation of the sterol ester compositions. Because of the instability of these compositions, their use in food products leads to food products with a shorter shelf-life. Additionally, although PUFAs have been shown to lower overall serum cholesterol levels, PUFAs also lower the “good” cholesterol (HDL) in addition to the “bad” (LDL) cholesterol.

Because past attempts have not provided wholly satisfactory solutions, the cholesterol lowering benefits of sterol esters have not been fully exploited. Accordingly, it would be desirable to provide sterol ester compositions having increased solubility but without a high level of PUFAs comprising the fatty acid moieties. It would also be desirable to provide food products, especially clear cooking and salad oils, comprising an efficacious amount of the sterol ester compositions.

### SUMMARY OF THE INVENTION

The present invention provides sterol ester compositions having increased solubility but without a high level of PUFAs comprising the fatty acid moieties. The sterol ester compositions of the present invention have fatty acid moieties comprising more than 50% monounsaturated fatty acids (MUFAs), preferably from about 55% to about 80% MUFAs, and more preferably from about 60% to about 75% MUFAs. Preferably, the fatty acid moieties comprise less than about 6% saturated fatty acids (SFAs), more preferably from about 0.1% to about 4% SFAs, and most preferably from about 0.5% to about 2% SFAs. The fatty acid moieties of the sterol ester compositions comprise 50% or less PUFAs.

Unlike PUFAs, MUFAs have been shown to lower “bad” cholesterol (LDL) while maintaining or raising “good” cholesterol (HDL).

The sterol ester compositions can be added in sufficient quantity to food products to provide enhanced hypocholesterolemic properties. When added to a clear edible oil such as a cooking or salad oil, the oil remains clear and uncloudy. Other food products to which the sterol ester compositions may be added include, but are not limited to, shortening, peanut butter, peanut spread, mayonnaise, sauces, gravies, margarine, health bars, snacks, beverages, ice cream, yogurt, cake mix, frosting, donuts, baked goods (e.g., breads and muffins), cheese, and cheese spreads. Additionally, the sterol ester compositions may be delivered in capsule form.

The preferred cooking or salad oil of the present invention comprises an edible oil and from about 5% to about 30%, preferably from about 10% to about 20%, sterol ester composition (calculated on a sterol ester basis). Preferably, the edible oil is a clear (uncloudy) edible oil.

In general, an efficacious amount of sterol ester to be delivered by the present invention is about 1.8 g of sterol ester per serving or dose, with 1-2 servings or doses per day.

Furthermore, the invention relates to a method for preparing the sterol ester compositions. The method comprises the steps of:

(a) providing fatty acid esters;

(b) preparing a sterol ester mixture;

(c) refining the sterol ester mixture;

(d) evaporating and stripping the sterol ester mixture; and

(e) fractionating the sterol ester mixture to obtain a sterol ester composition.

Optionally, but preferably, a nucleating agent is added before the fractionating step.

### DETAILED DESCRIPTION OF THE INVENTION

Definitions

As used herein, the term “hypocholesterolemic” means reducing the cholesterol level, or inhibiting or reducing the build-up of cholesterol, in the blood of warm-blooded animals.

As used herein, the term “HDL” means high-density lipoprotein.

As used herein, the term “LDL” means low-density lipoprotein.

As used herein, the term “plant sterol” includes all non-animal sterols, including not only phytosterols (plant sterols characteristic of higher plants), but also mycosterols (plant sterols from lower plants). For a more complete description of plant sterols, see Deuel, The Lipids, New York: Interscience Publishers (Volume 1, 1951), at pages 321 and 348.

As used herein, the term “sterol” includes sterols, stanols (the ring-saturated derivatives of sterols), and mixtures thereof.

As used herein, the term “sterol ester” includes sterol esters, stanol esters, and mixtures thereof.

As used herein, the term “ester” refers to carboxylic acid ester.

As used herein, the term “room temperature” means a temperature of about 20° C.

As used herein, the term “edible oil” refers to a pourable (at room temperature) fatty substance that is suitable for consumption. Edible oils can include natural and synthetic oils, including non-digestible oils, and mixtures thereof.

As used herein, the term “non-digestible” refers to materials that are partially or totally indigestible, e.g., polyol fatty acid polyesters.

All percentages herein are by weight unless stated otherwise.

All percentages of sterol esters are by weight on a sterol ester basis (rather than on a free sterol basis).

Preparation of the Sterol Ester Compositions

The preferred method for making the sterol ester compositions of the present invention is set forth in detail below.

A. Providing Fatty Acid Esters

As used herein, the term “fatty acid esters” is intended to include the C₁-C₄ (preferably methyl), 2-methoxy ethyl and benzyl esters of fatty acids containing about eight or more carbon atoms, and mixtures of such esters. Suitable reactant esters can be prepared by the reaction of diazoalkanes and fatty acids, or derived by alcoholysis from the fatty acids naturally occurring in fats and oils. For example, methanol and sodium methylate catalyst can be added to a triglyceride and heated to reflux with stirring for 1-4 hours. The reaction mixture is allowed to settle without agitation and the glycerin layer is removed. Additional methanol and sodium methylate catalyst are added and the mixture is again heated to reflux for another 1-4 hours. The mixture is allowed to settle without stirring and the glycerin layer removed. The crude methyl ester is then washed with water, dried and fractionally distilled. This process is described in U.S. Pat. No. 5,491,226 issued Feb. 3, 1996 to Kenneally.

Suitable fatty acid esters can be derived from either synthetic or natural fatty acids and can include positional and geometrical isomers. Suitable preferred saturated fatty acids include, for example, caprylic, capric, lauric, isomyristic, isomargaric, and anteisoarachadic. Suitable preferred unsaturated fatty acids include, for example, myristoleic, palmitoleic, ricinoleic, linoleic, oleic, elaidic, eleostearic, arachidic, arachidonic, erucic, and erythrogenic acids.

Mixtures of fatty acids derived from olive oil, high-oleic sunflower oil mid-oleic sunflower seed oil, high-oleic safflower oil, sesame seed oil, peanut oil, rice bran oil, and canola oil are especially preferred for use herein. C₁₆-C₁₈ fatty acids can be provided by soybean oil or cottonseed oil. Shorter chain fatty acids can be provided by coconut, palm kernel, or babassu oils. Corn oil, olive oil, palm oil, peanut oil, safflower seed oil, sesame seed oil, and sunflower seed oil, are examples of other natural oils which can serve as the source of the fatty acid component. Any other suitable source of fatty acids can also be used.

B. Preparing a Sterol Ester Mixture

The plant sterol carboxylic acid esters can have their sterol moieties derived from any plant sterol. For example, the sterol moieties can be derived from plant sterols such as, for example, β-sitosterol, stigmasterol, or campesterol. The sterol moieties can also be derived from stanols (the ring-saturated derivatives of sterols) such as β-sitostanol, stigmastanol, or campestanol. The stanols can be naturally occurring or derived from hydrogenation of sterols. The sterol moieties can also be derived from mixtures of these plant sterols and stanols, such as those found as natural components of soy or canola oil.

Plant sterol carboxylic acid esters can be derived from commercially available plant sterols by any convenient acylation method. For example, plant sterol monocarboxylic acid esters can be prepared by perchloric acid catalyzed esterification of the free sterols with monocarboxylic acid anhydrides. See, e.g., British Patent GB 1,405,346 to Baltes et al., published Sep. 10, 1975. U.S. Pat. No. 5,219,733 to Myojo et al. describes a process for preparing sterol fatty acid esters with the use of enzymes. Various other methods for preparing sterol fatty acid esters are described by A. Kuksis et al. in “Preparation and Certain Physical Properties of Some Plant Steryl Esters,” J. Org. Chem. 25:1209-19 (1960).

A preferred method for preparing sterol esters comprises reacting the sterols with lower alkyl fatty acid esters of the desired composition in the presence of a basic catalyst to form a crude sterol ester reaction mixture (the “crude”). The lower alkyl esters serve a dual role of being a reactant and of acting as the solvent. The basic catalyst can be any known interesterification catalyst such as sodium methoxide. The reactions are run under vacuum or with inert gas sparging or combinations thereof to remove methanol as it is formed and force the reaction to higher degrees of esterification. This process is described in U.S. Pat. No. 5,985,913, issued Sep. 28, 1999 to Miettenen. A small amount of water is then added to the crude to hydrate any soaps that have formed as the result of side reactions. The hydrated soap is removed by centrifugation or filtration or combinations thereof. The crude can be washed with water. The crude is then dried and bleached by conventional oil processing techniques. Silica gel is a preferred bleaching agent. Residual methyl ester is removed by vacuum distillation at elevated temperatures such as on a wiped film evaporator. The sterol ester is then deodorized by conventional techniques. Steam is a preferred stripping agent.

Preferably, the degree of esterification of the sterols is at least 90%. In one embodiment, the sterol ester composition comprises less than about 10% free sterols, preferably less than about 5% free sterols, and more preferably less than about 3% free sterols.

The basic catalysts generally suitable for use in preparing the sterol esters described herein are those selected from the group consisting of alkali metals, such as aluminum, sodium, lithium and potassium: alloys of two or more alkali metals, such as sodium-lithium and sodium-potassium alloys; alkali metal hydrides, such as sodium, lithium and potassium hydride; and alkali metal alkoxides, such as potassium t-butoxide and sodium methoxide. The use of these catalysts is further taught in U.S. Pat. No. 4,517,360, issued May 14, 1985 to Volpenhein.

More reactive catalysts such as potassium or sodium methoxide should be protected until their addition into the reaction mixture. Preferably the catalyst should be suspended in, or more preferably encapsulated by, a material that will either be present in the reaction mixture or be readily separated from the reaction mixture. Suitable encapsulating agents include alkyl esters of, for example, C₁₆-C₂₂ fatty acids. The use of these encapsulating agents is further taught in U.S. Pat. No. 5,124,301, issued Jun. 23, 1992 to Wyness.

Although the use of saturated fatty acid methyl ester encapsulation agents introduces more saturated, potentially insoluble material into the crude, the amount is essentially insignificant and most of the resulting saturated fatty acid sterol esters can subsequently be removed through winterization.

In general, an initial heterogeneous crude reaction mixture comprises from about 40% to about 60%, preferably from about 45% to about 55%, by weight of sterol; from about 60% to about 40%, preferably from about 55% to about 45%, by weight of the fatty acid lower alkyl esters; and from about 0.1% to about 3%, preferably from about 0.1% to about 1%, by weight of basic catalyst component. It can be desirable to add more basic catalyst toward the end of the reaction. The catalyst in the initial step can be potassium carbonate as described above or an alkali metal hydroxide at low levels or, most preferred, potassium or sodium methoxide. Solutions of potassium or sodium methoxide in methanol can be used as the initial catalyst; however, any re-catalysis should be performed using solid, dry potassium or sodium methoxide.

The reaction mixture is typically heated to a temperature within the range of from about 194° F. (90° C.) to about 325° F. (163° C.), preferably from about 266° F. (130° C.) to about 284° F. (140° C.), under a pressure of from about 0.1 mm Hg to about 760 mm Hg. The reaction mixture is agitated (e.g., stirred). In addition, the mixing is increased by sparging with an inert gas, preferably nitrogen, carbon dioxide, low molecular weight hydrocarbons, or oxides of nitrogen. With sparging, the removal of volatile alcohol produced in the reaction is promoted and the reaction speed is increased such that the temperature can be kept low and/or the pressure can be kept higher. Low temperatures are highly desirable to minimize the formation of unwanted by-products including beta-ketoesters and di-fatty ketones, other carbonyl compounds, ring structures, and dehydrated sterols.

C. Refining, Evaporating, and Stripping the Sterol Ester Mixture

After the reaction has reached the desired state of completion, the catalyst, the residual fatty ester reactant, and any soap (formed as a by-product from residual moisture in the raw materials or from the dehydration of sterols) are removed, since they should not be consumed along with the sterol ester. This removal is accomplished during the refining stage and finishing stage (evaporating and stripping) of sterol ester manufacture. 1. Refining the Sterol Ester Mixture

Refining of the sterol ester comprises removing the soap and catalyst from the crude product. Suitable refining steps are disclosed in Erickson, World Conference Proceedings, “Edible Fats and Oils Processing,” American Oil Chemists Soc. (1990). Most refining methods primarily involve adding water to the crude sterol ester, and subsequently removing it by centrifuging the mixture. This method is effective for removing soap and catalyst. The level of water added to the crude sterol ester is from about one half to about ten times the amount of soap removed by the method. However, even after centrifuging, the reaction mixture can still contain an undesirable level of residual soap and/or color bodies. Therefore, it is sometimes useful to repeat the water washing step followed by gravity or centrifugal separation of the aqueous phase.

A subsequent refining step involves a vacuum drying and adsorptive bleaching operation. This step can be used in combination with, or in place of, the second washing step above. Adsorbents such as bleaching earth, silica gel, and activated charcoal are typically used in drying and/or adsorptive bleaching operations of edible oils. The adsorbents are preferably added at a level of from about 0.1% to about 10% by weight of the dry reaction mixture. After the bleaching operation, the adsorbents are removed from the reaction mixture by centrifugation or filtration. The second stage water washing, and/or drying, and/or adsorptive bleaching completes the removal of soap and color bodies.

2. Evaporating and Stripping the Sterol Ester Mixture

Evaporating and stripping the sterol ester mixture comprises removing unwanted materials such as free fatty acids, excess fatty acid ester reactant, and negative flavor components. Typical fatty acid removal is disclosed in Cowan, Degumming, Refining, Bleaching, and Deodorization Theory, 53 Journal of American Oil Chemists Soc. (June 1976). The evaporating and stripping steps (“finishing steps”) used in the present invention can include thermal evaporation, high temperature steam distillation, or combinations thereof.

a. Thermal Evaporation

Thermal evaporation comprises heating the sterol ester crude to a temperature equivalent to the unwanted material's bubble point at evaporator pressure. The crude is fed into a thermal evaporator, such as agitated film, wiped film, flash, rising film, or falling film evaporator, wherein the crude is heated to a temperature of from about 380° F. (193° C.) to about 550° F. (288° C.) at an absolute pressure of about 0.2 mm Hg to about 5 mm Hg to remove the bulk of the unwanted materials.

b. High Temperature Steam Distillation

High temperature steam distillation comprises deaerating the sterol ester to a level of less than about 0.10% by volume of dissolved oxygen, heating the deaerated oil to a temperature between about 390° F. (199° C.) to about 525° F. (273° C.), and stripping the oil at an absolute pressure of less than about 15 mm Hg for a period of from about 5 minutes to about 2.5 hours using a medium such as steam, nitrogen, or an inert gas in an amount from about 0.2% to about 20% by weight of the sterol ester.

The finished sterol fatty acid ester may be further treated with color removing adsorbent such as silica gel and subsequently deodorized if needed.

D. Fractionating the Sterol Ester Mixture to Obtain a Sterol Ester Composition

1. Winterization or Crystal Fractionation

Winterization or crystal fractionation of solid sterol esters can be carried out with or without solvents, with or without agitation, and with or without heating or cooling above or below ambient temperature. Nucleation aids such as bleaching earths (such as Filtrol™, a diatomaceous earth made by the Filtrol Corporation) or filter aids (such as Celite™, a diatomaceous earth produced by World Minerals, Inc. of Santa Barbara, Calif.) can be added prior to crystallization to speed up the process and aid the formation of large, easily filtered crystals. The crystal fractionation can be repeated several times.

a. Solvent Process

In a preferred embodiment, vegetable oil is used as the solvent and the crystallization is carried out in a concentrated solution at ambient temperature, insoluble solids are removed by conventional techniques, and the final product is obtained by further diluting the clear concentrate with additional vegetable oil. Vegetable oils containing desired sterol fatty acid esters can be obtained by dissolving 10-60% sterol fatty acid ester, preferably 40-60% sterol fatty acid ester of the composition of this invention, in the vegetable oil of choice for the final product. The sterol ester vegetable oil blend is heated and mixed to totally dissolve the sterol esters and then allowed to cool to ambient temperature. The blend is placed in a 70° F. (21° C.) constant temperature room for 12-72 hours, preferably 24-48 hours, and the insoluble sterol esters are allowed to precipitate. The solids can be separated from the solution of soluble sterol esters by centrifugation or filtration or combinations thereof. If filtration is used, care should be taken to minimize the amount of shear to which the blend is exposed in order to maintain large, easily filterable crystals. The resulting clear oil is analyzed by the carbon number profile GC method to determine the concentration of sterol ester in the winterized concentrate, and then is diluted with additional vegetable oil to obtain the desired final concentration of product. It is desirable to conduct the entire winterization process under an inert atmosphere such as under a nitrogen blanket.

In another embodiment, a small amount of a nucleation aid is added to the previously described process, either before the initial blend of sterol ester and vegetable oil is heated to effect dissolution, or after the sterol esters are totally dissolved. The nucleation aid can be any vegetable oil insoluble solid such as Celite™, Filtrol™, hardstock triglyceride, or previously isolated saturated fatty acid sterol esters. Most preferred are the previously isolated saturated fatty acid sterol esters. The nucleation aid can be used at a level of from about 0.1% to about 5% by weight of the oil blend, preferably from about 0.2% to about 0.5% of the blend.

When using a filter paper or a filter press, it is desirable to pre-coat the paper or the press with a filter aid such as Celite™. This prevents the filter from blinding.

It is sometimes desirable to perform the crystallization at lower than ambient temperatures. This is especially so when utilizing more diluted concentrations of sterol ester in vegetable oil, such as where sterol esters are present at a concentration of from about 5% to about 30%, preferably from about 10% to about 20%. Vegetable oils containing desired sterol fatty acid esters can be obtained by dissolving 5-30% sterol fatty acid ester, preferably 10-20% sterol fatty acid ester of the composition of this invention in the vegetable oil of choice for the final product. The sterol ester vegetable oil blend is heated and mixed to totally dissolve the sterol esters and then allowed to cool to ambient temperature. The blend is then processed by a procedure similar to the one described in “Bailey's Industrial Oil And Fat Products,” 3rd Ed., New York: Interscience Publishers (D. Swem ed., 1964), at pages 1008-1009. The blend at a temperature of 70° F. (21° C.) to 80° F. (27° C.) is cooled to 55° F. (13° C.) over the course of 6-12 hours. Crystallization begins at this temperature. The blend is then cooled to 45° F. (7° C.) over the course of an additional 12-18 hours. The blend is held at this temperature until the desired degree of winterization has occurred, usually about 12 hours. The solids can be separated from the solution of soluble sterol esters by centrifugation or filtration or combinations thereof. If filtration is used, care should be taken to minimize the amount of shear to which the blend is exposed in order to maintain large, easily filterable crystals. The resulting clear oil is analyzed by the carbon number profile GC method to determine to determine the concentration of sterol ester in the winterized concentrate, and then is diluted with additional vegetable oil to obtain the desired final concentration of product. It is desirable to conduct this entire process under an inert atmosphere such as under a nitrogen blanket.

In another preferred embodiment, a food approved organic solvent other than triglyceride (such as hexane, mixed hexanes, methanol, acetone or mixtures thereof) is used as the solvent and the crystallization is performed in a concentrated solution at ambient temperature; the insoluble solids are then removed from the solution of soluble sterol esters and the solvent stripped from the soluble sterol ester solution by conventional techniques. Vegetable oils containing desired sterol fatty acid esters can be obtained by dissolving 10-60%, preferably 25-50%, sterol fatty acid ester of the composition of this invention in a solvent, preferably food grade hexane. The sterol ester-hexane blend is heated and mixed to totally dissolve the sterol esters and then allowed to cool to ambient temperature. The blend is cooled to −15 to 30° C., preferably between −10 to 10° C. for 6-48 hours, and more preferably 12-24 hours, and the insoluble sterol esters are allowed to precipitate. The solids can be separated from the solution of soluble sterol esters by centrifugation or filtration or combinations thereof. If filtration is used, care should be taken to minimize the amount of shear to which the blend is exposed in order to maintain large, easily filterable crystals. The solvent is removed from the resulting clear soluble sterol ester solution by distillation or on a wiped film evaporator. To remove any residual solvent, the product can be placed in a vacuum oven for a period of 1-6 hours at a temperature of 20-60° C. and a pressure of 1-25 mm Hg. The soluble sterol ester is then added to vegetable oil in the desired concentration. The sterol ester-vegetable oil blend is heated and mixed to totally dissolve the sterol esters and then allowed to cool to ambient temperature. It is desirable to conduct this entire process under an inert atmosphere such as under a nitrogen blanket.

b. Solventless Process

Winterization or crystal fractionation of sterol esters can be carried out without the use of solvents. This is generally performed from ambient temperature to slightly elevated temperatures to decrease the viscosity of the soluble phase and make centrifugation and/or filtration easier, but always below the melting point of the solid sterol esters. In a preferred embodiment the sterol ester is completely melted and then allowed to crystallize at ambient temperature, insoluble solids removed by conventional techniques, and the final product is obtained by adding the clear liquid sterol ester at the desired concentration to vegetable oil.

Vegetable oils containing desired sterol fatty acid esters can be obtained by heating and mixing the neat sterol fatty acid ester of the composition of this invention to 120° F. (49° C.) to 160° F. (71° C.) to totally melt and/or dissolve the sterol esters and then allowing the blend to cool to ambient temperature. The blend is placed in a 85° F. (29° C.) constant temperature room for 12-72 hours, preferably 24-48 hours and the insoluble sterol esters are allowed to precipitate. The solids can be separated from the liquid phase by centrifugation or filtration or combinations thereof. If filtration is used, care should be taken to minimize the amount of shear to which the blend is exposed in order to maintain large, easily filterable crystals. The soluble sterol ester is then added to vegetable oil in the desired concentration. The sterol ester-vegetable oil blend is heated and mixed to totally dissolve the sterol esters and then allowed to cool to ambient temperature. It is desirable to conduct this entire process under an inert atmosphere such as under a nitrogen blanket.

In another embodiment, a small amount of a nucleation aid is added to the previously described solventless process, either before the initial blend of sterol ester and vegetable oil is heated to effect dissolution, or after the sterol esters are totally melted and/or dissolved prior to the crystallization step. The nucleation aid can be any vegetable oil insoluble solid such as Celite™, Filtrol™, hardstock triglyceride, or previously isolated saturated fatty acid sterol esters. Most preferred are previously isolated saturated fatty acid sterol esters. The nucleation aid can be used at a level of 0.1% to 5% by weight of the oil blend, preferably at a level of 0.2% to 0.5% of the oil blend. It is removed during the centrifugation or filtration.

2. Fractionation of the Starting Methyl Esters

An alternate way to achieve low levels of saturates in the finished sterol ester is to start with lower alkyl esters that are themselves low in saturated fatty acid chain lengths. It is possible to remove a substantial amount of the C₁₆ saturated fatty acid lower alkyl esters (palmitic acid esters) by fractional distillation. It is much more difficult to reduce the level of the C₈ saturated fatty acid lower alkyl esters (stearic acid esters) because of the similarity in boiling points with C18:1, C18:2 and C18:3 unsaturated fatty acid lower alkyl esters (oleic, linoleic and linolenic acid esters). In order to have a finished sterol ester that contains greatly reduced levels of saturated fatty chains, it is necessary to select a feedstock in which the majority of the saturation is in the form of saturated C₁₆ fatty acid lower alkyl esters. As it is also desirable to limit the amount of polyunsaturated fatty acid lower alkyl esters, only certain feedstocks meet these criteria. Preferred feedstocks include olive, canola, peanut, high-oleic safflower, high-oleic sunflower, mid-oleic sunflower, rice bran, and canola lower alkyl esters.

Distillations can be performed either batch or continuously using fractionation columns that contain any of the commercially available structured solid packings. It is desirable that the distillations be performed at the lowest possible temperatures and lowest pressures to prevent thermal decomposition. When the lower alkyl esters are methyl esters, preferred temperatures are in the range of 350° F. (177° C.) to 450° F. (232° C.) and preferred pressures are in the range of 0.5 to 20 mm Hg, more preferably from 2 to 15 mm Hg. A top cut is taken which is rich in saturated C₁₆ lower alkyl esters. The amount of the topcut depends of the type of feedstock and how much of the C₁₆ lower alkyl esters are removed. After taking a topcut, the desired low saturated C₁₆ lower alkyl esters are collected. High boiling point material is left as still bottoms. The amount depends on the type of feedstock. The still bottoms may be recycled by combining them with the feedstock for future distillations.

Fractional distillation of the starting methyl esters can be used alone or in combination with winterization or any of the other fractionation techniques described herein.

3. Argentation Chromatography

Sterol esters can also be fractionated by argentation chromatography including industrial scale, continuous chromatography as described in U.S. Pat. No. 4,297,292, issued Oct. 27, 1981 to Logan. The macroreticular strong acid cation exchange resins substituted with 20-80% silver ions (+1 oxidation state) are suitable as the stationary phase. The unsaturated fatty acid sterol esters are more strongly retained on the resin and can thus be separated from the more weakly retained saturated fatty acid sterol esters.

4. Fractionation of Fatty Acids

Removal of saturated fatty chain lengths can be accomplished from fatty acids as well as from methyl esters or sterol esters. The fatty acids can be fractionated by known conventional techniques such as a hydrophilization process (Henkel process) described in “Bailey's Industrial Oil And Fat Products,” 4th Ed., Vol. 11, New York: Wiley-Interscience (D. Swem ed., 1982), at pages 381-382. Fatty acids can also be fractionally distilled as described at id., pages 382-384. It is possible to remove a substantial amount of the C₁₆ saturated fatty acid (palmitic acid) by fractional distillation. It is much more difficult to reduce the level of the C₁₈ saturated fatty acid (stearic acid) because of the similarity in boiling points with C18:1, C18:2 and C18:3 unsaturated fatty acids (oleic, linoleic and linolenic acids). In order to produce a finished sterol ester that contains greatly reduced levels of saturated fatty chains, it is necessary to select a feedstock in which the majority of the saturation is in the form of saturated C₁₆ fatty acid. As it is also desirable to limit the amount of polyunsaturated fatty acid lower alkyl esters, only certain feedstocks meet these criteria. Preferred feedstocks include olive, canola, peanut, high oleic safflower, high oleic sunflower, mid oleic sunflower, rice bran and canola fatty acids.

The resulting predominantly unsaturated fatty acids resulting from either the Henkel process or fractional distillation can be reacted directly with the sterol in an acid catalyzed reaction or converted into lower alkyl esters such as methyl esters and reacted with sterol in the presence of a basic catalyst as described previously herein.

E. Characteristics of the Sterol Ester Compositions

The resulting sterol ester compositions have fatty acid moieties comprising more than 50% monounsaturated fatty acids (MUFAs), preferably from about 55% to about 80% MUFAs, and more preferably from about 60% to about 75% MUFAs. Preferably, the fatty acid moieties comprise less than about 6% saturated fatty acids (SFAs), more preferably from about 0.1% to about 4% SFAs, and most preferably from about 0.5% to about 2% SFAs. The fatty acid moieties of the sterol ester compositions comprise 50% or less PUFAs.

The Cooking or Salad Oil

Although the end use of the sterol ester compositions is described primarily herein in terms of a preferred cooking or salad oil, it should be readily apparent to one skilled in the art that the sterol ester compositions can be used in the preparation of any suitable product. For instance, the sterol ester compositions can be added to food products such as shortening, peanut butter, peanut spread, mayonnaise, sauces, gravies, margarine, health bars, snacks, beverages, ice cream, yogurt, cake mix, frosting, donuts, baked goods (e.g., breads and muffins), cheese, and cheese spreads. The sterol ester compositions can be added in sufficient quantity to food products to provide enhanced hypocholesterolemic properties. Additionally, the sterol ester compositions may be delivered in capsule form.

In general, an efficacious amount of sterol ester to be delivered by the present invention is about 1.8 g of sterol ester per serving or dose, with 1-2 servings or doses per day.

The preferred cooking or salad oil of the present invention comprises an edible oil and from about 5% to about 30%, preferably from about 10% to about 20%, sterol ester composition (calculated on a sterol ester basis). Preferably, the edible oil is a clear edible oil. When the sterol ester composition is added to such a clear edible oil, the oil remains clear and uncloudy. The preferred salad or cooking oil comprises more than about 10% sterol ester composition and is free of solids at temperatures of greater than about 60° F. (16° C.).

The sterol ester composition may be admixed with the edible oil by any suitable means, such as by blending, shaking or stirring, to form the cooking or salad oil of the present invention.

As used herein, the term “edible oil” refers to a pourable (at room temperature) fatty substance that is suitable for consumption. Edible oils can include natural and synthetic oils, including non-digestible oils, and mixtures thereof.

A wide variety of clear glyceride oils can be used in the compositions of this invention. Pure triglycerides liquid at normal home refrigerator temperatures, such as triolein, are especially suitable. The preferred suitable oils are the so called natural salad oils such as, for example, olive oil, high-oleic sunflower seed oil, mid-oleic sunflower seed oil, high-oleic safflower oil, sesame seed oil, peanut oil, rice bran oil, and canola oil. Preferably, these preferred oils are refined, bleached, and deodorized, but not hardened or winterized. Other naturally occurring liquid glyceride oils can be used such as cottonseed oil and corn oil; these oils can be given a preliminary “winterization,” dewaxing, or similar treatment to remove the higher melting stearines before being used as an oil base. Certain other oils such as soybean oil can be partially hydrogenated before use to improve their resistance to oxidative deterioration during prolonged storage periods; the higher melting solids formed during the hydrogenation treatment are preferably removed by winterization.

Suitable clear glyceride oils can also be obtained by directed, low temperature interesterification or rearrangement of animal or vegetable fatty materials, followed by removal of the higher melting solids formed during the reaction. For an example of this procedure, see U.S. Pat. No. 2,442,532. Another group of oils suitable for use as the liquid glyceride oil is that group of oils in which one or more short-chain fatty acids, such as acetic acid or propanoic acid, replace in part the long chain fatty acids present in natural triglyceride oils. Also suitable are diglycerides, such as those diglycerides disclosed in U.S. Pat. No. 4,976,984, issued Dec. 11, 1990 to Yasukawa, and those disclosed by Tsutomu in Japanese Published Application JP 090282889 A, published Feb. 4, 1997.

Any other useful clear glyceride oils derived from animal, vegetable or marine sources, including mixtures thereof of such oils, may also be used.

Low calorie and zero calorie oil substitutes, such as sucrose polyesters of long chain fatty acids (olestra) and other polyol polyesters of fatty acids, can also be used as the edible oil herein. See, e.g., U.S. Pat. No. 3,600,186 to Mattson et al.; U.S. Pat. No. 5,422,131 to Elsen et al.; U.S. Pat. No. 5,419,925 to Seiden et al.; and U.S. Pat. No. 5,071,669 to Seiden. Mixed triglycerides made from medium and long chain saturated and/or unsaturated fatty acids can also be used as the edible oil herein. See, e.g., U.S. Pat. No. 5,288,512 to Seiden. Oils that contain medium chain triglycerides can also be used. See, e.g., U.S. Pat. No. 4,863,753 to Hunter et al.

Non-digestible oils include liquid polyol fatty acid polyesters (see, e.g., U.S. Pat. No. 4,005,195, issued Jan. 25, 1977 to Jandacek); liquid esters of tricarballylic acids (see, e.g., U.S. Pat. No. 4,508,746, issued Apr. 2, 1985 to Hamm); liquid diesters of dicarboxylic acids such as derivatives of malonic and succinic acid (see, e.g., U.S. Pat. No. 4,582,927, issued Apr. 15, 1986 to Fulcher); liquid triglycerides of alpha-branched chain carboxylic acids (see, e.g., U.S. Pat. No. 3,579,548, issued May 18, 1971 to Whyte); liquid ethers and ether esters containing the neopentyl moiety (see, e.g., U.S. Pat. No. 2,962,419, issued Nov. 29, 1960 to Minich); liquid fatty polyethers of polyglycerol (see, e.g., U.S. Pat. No. 3,932,532, issued Jan. 13, 1976 to Hunter et al); liquid alkyl glycoside fatty acid polyesters (see, e.g., U.S. Pat. No. 4,840,815, issued Jun. 20, 1989 to Meyer et al); liquid polyesters of two ether linked hydroxypolycarboxylic acids (e.g., citric or isocitric acid) (see, e.g., U.S. Pat. No. 4,888,195, issued Dec. 19, 1988 to Huhn et al); various liquid esterified alkoxylated polyols including liquid esters of epoxide-extended polyols such as liquid esterified propoxylated glycerins (see, e.g., U.S. Pat. No. 4,861,613, issued Aug. 29, 1989 to White et al; U.S. Pat. No. 5,399,729, issued Mar. 21, 1995 to Cooper et al; U.S. Pat. No. 5,589,217, issued Dec. 31, 1996 to Mazurek; and U.S. Pat. No. 5,597,605, issued Jan. 28, 1997 to Mazurek); liquid esterified ethoxylated sugar and sugar alcohol esters (see, e.g., U.S. Pat. No. 5,077,073 to Ennis et al); liquid esterified ethoxylated alkyl glycosides (see, e.g., U.S. Pat. No. 5,059,443, issued Oct. 22, 1991 to Ennis et al); liquid esterified alkoxylated polysaccharides (see, e.g., U.S. Pat. No. 5,273,772, issued Dec. 28, 1993 to Cooper); liquid linked esterified alkoxylated polyols (see, e.g., U.S. Pat. No. 5,427,815, issued June 27, 1995 to Ferenz; and U.S. Pat. No. 5,374,446, issued Dec. 20, 1994 to Ferenz et al.); liquid esterified polyoxyalkylene block copolymers (see, e.g., U.S. Pat. No. 5,308,634, issued May 3, 1994 to Cooper); liquid esterified polyethers containing ring-opened oxolane units (see, e.g., U.S. Pat. No. 5,389,392, issued Feb. 14, 1995 to Cooper); liquid alkoxylated polyglycerol polyesters (see, e.g., U.S. Pat. No. 5,399,371, issued Mar. 21, 1995 to Harris); liquid partially esterified polysaccharides (see, e.g., U.S. Pat. No. 4,959,466, issued Sep. 25, 1990 to White); as well as liquid polydimethyl siloxanes (e.g., fluid silicones available from Dow Coming™). Solid non-digestible fats or other solid materials can be added to the liquid non-digestible oils to prevent passive oil loss. Particularly preferred non-digestible fat compositions include those described in U.S. Pat. No. 5,490,995 issued to Corrigan; U.S. Pat. No. 5,480,667 issued to Corrigan et al; U.S. Pat. No. 5,451,416 issued to Johnston et al.; and U.S. Pat. No. 5,422,131 issued to Elsen et al. U.S. Pat. No. 5,419,925 issued to Seiden et al. describes mixtures of reduced calorie triglycerides and polyol polyesters that can be used herein. Any other suitable non-digestible oils, reduced calorie oils, oil substitutes, or glyceride oils may be used herein.

Suitable mixtures of any of the oils described herein may also be used.

### ANALYTICAL TEST METHOD

Carbon Number Profile GC Method

AOCS (American Oil Chemists Society) Analytical Method Ce5-86, “Triglycerides by Gas Chromatography.”

### EXAMPLES

The following examples are illustrative of the present invention but are not meant to be limiting thereof.

### Example 1

### Preparation of Canola Methyl Esters

Add Crisco™ Brand canola oil (3451 grams) to a 10 liter, 3-neck glass reactor equipped with nitrogen inlet, turburane stirrer and reflux condenser. Purge headspace with nitrogen for 2 hours. Add methanol (667 grams) and sodium methylate (50 grams), and heat to reflux temperature and allow to reflux for 1 hour. Let reaction mixture settle for 20 minutes and draw off the glycerin layer (bottom). Add methanol (178 grams) and sodium methylate (29 grams) and heat to reflux temperature again. Let reaction mixture reflux for 2 additional hours. Let reaction mixture settle for 20 minutes and draw off the glycerin layer (bottom). Allow mixture to cool to 35° C. and add 400 ml of tap water. Agitate for 5 minutes and then transfer entire contents to centrifuge tubes and centrifuge in batches until emulsion breaks and phases are separate. Pour off the crude ester layer (top) and put half of it back into the reactor. Replace the reflux condenser with a 20 inch glass column (2 inch diameter (5 cm)) packed with structured glass packing material (8 mm x 8 mm hollow cylinders), reduce the pressure to about 1-3 mm Hg, and heat until material begins to distill over. Discard the first 50 ml distillate and collect the next 1450 grams. Repeat the distillation with the second half of the crude esters and collect 1480 grams of distilled ester. The collected distillate is canola methyl esters.

### Example 2

### Preparation Of Canola Fatty Acid Sterol Esters

Add canola methyl esters from Example 1 (2890 grams) and mixed sterols (3100 grams) which are predominantly soy sterols (such as those available from ADM, Decatur, Ill.) to a 50 liter, 3-neck glass reactor equipped with nitrogen inlet, turburane stirrer and distillation head. Purge headspace with nitrogen for 2 hours. Initiate agitation and apply vacuum (10 mm Hg). Heat this mixture to 115° C., adjusting the pressure as needed to control bubbling and foaming. When no more water appears in the collection flask, bring the reaction mixture back to atmospheric pressure and slowly add sodium methylate solution (123 grams of 25% solution in methanol). Reconnect the vacuum and slowly reduce the pressure, adjusting as needed to control foaming. Heat the reaction mixture at 100° C. for 0.5 hours, then increase the temperature to 125° C. for 1.5 hours, and finally heat at 135° C. for 3 hours. The methanol formed as a result of the transesterification is continuously removed. Break the vacuum and bring back to atmospheric pressure under nitrogen purge. Let cool to room temperature under nitrogen. A sample is taken and analyzed by GC. This crude sterol ester reaction mixture contains 22.1% residual methyl ester, 2.0% free sterol, and 75.9% sterol ester.

The crude sterol ester reaction mixture is transferred in equal portions to 3 ½ gallon centrifuge jars. Water (30 ml) is added to each jar. The jars are capped and mixed by inverting 25 times. The jars are centrifuged for 5 minutes at 4000 rpm to separate the layers and the sterol ester layer (top) is decanted back into a clean 22 liter reactor. Silica (126 grams) is added and the mixture stirred for 2 hours at 85° C. with nitrogen purge. The mixture is transferred in equal protions to 3 ½ gallon centrifuge jars and centrifuged for 6 minutes at 4000 rpm. The centrifuged sterol ester is decanted (top phase) and filtered with a Buchner funnel fitted with Whatman 40 filter paper to remove the last traces of silica. The residual methyl esters are removed on a Pope wipped film evaporator operated at 220° C. and 1.5 mm Hg at a drip rate of 0.25 liters/hour.

The stripped sterol ester is deodorized in 450 gram batches in a 5 liter deodorizer operated at 500° F. (260° C.) at a pressure of 4 mm Hg for 2 hours. The final product is analyzed by GC and the composition found to contain 96.9% total sterol esters (90.2% unsaturated, 6.7% saturated) and 3.1% free sterols.

### Example 3

### Winterization of the Sterol Ester Using Vegetable Oil as the Solvent

Sterol ester from Example 2 (250 grams) and Crisco™ Brand canola oil (250 grams) are heated with stirring under nitrogen purge until all the sterol ester dissolves in the canola oil. The mixture is cooled to room temperature, tightly stoppered and placed in a 70° F. (21° C.) constant temperature room for 16 hours during which time precipitation occurs. The mixture is transferred to a centrifuge ½ gallon centrifuge jar and centrifuged at 4000 rpm for 5 minutes. The resulting clear sterol layer (top phase) is removed by decantation. The clear sterol ester is again placed in a 70° F. constant temperature room for 24 hours. More precipitate is formed. This precipitate is also removed by centrifugation. A total of 213 grams of soluble product is isolated after the second centrifugation. This material (the sterol ester composition) is analyzed by GC and is found to contain 43.5% sterol esters (41.5% unsaturated fatty acid sterol esters, 2.0% saturated fatty acid sterol esters) and 2.0% free sterol, the remainder being vegetable oil. (The process is 87% efficient; 43.5% sterol ester results from the starting quantity of 50% sterol ester.)

### Example 4

### Winterization of the Sterol Ester Using Canola Oil as the Solvent and Celite™ as Nucleating Agent

Sterol ester from Example 2 (150 grams), Crisco™ Brand canola oil (100 grams) and Celite™ (0.18 grams) are heated with stirring under nitrogen purge until all the sterol ester dissolves in the canola oil. The mixture is cooled to room temperature, tightly stoppered and placed in a 70° F. (21° C.) constant temperature room for 24 hours during which time precipitation occurs. The mixture is filtered using a Buchner funnel and Whatman 40 filter paper precoated with Celite™ (made by running a slurry of Celite™ (5 grams) and Crisco™ Brand canola oil (50 grams) through the filter paper prior to the actual filtration of product). A total of 23.5 grams of soluble product is isolated after centrifugation. This material (the sterol ester composition) is analyzed by GC and is found to contain 40.1% sterol esters (38.2% unsaturated fatty acid sterol esters, 1.9% saturated fatty acid sterol esters) and 1.1% free sterol.

### Examples 5

### Winterization of the Sterol Ester Using Hexane as the Solvent

Sterol ester from Example 2 (10 grams) is weighed into a 50 ml Erlenmeyer flask. Hexane (20 ml) is added to the flask along with a magnetic stir bar. The flask is heated with stirring on a stirring hot plate at 50° C. until the sterol ester has completely dissolved in the solvent. The hexane solution is cooled to 25° C. under nitrogen and the flask is tightly stoppered. The flask containing the hexane solution is placed in a freezer at 0° C. for 16 hours. The precipitate is removed by filtration through a fine sintered glass filter. The hexane is removed from solute on a rotovap operated at a pressure of 25 mm Hg and a temperature of 50° C. The resulting unsaturated fatty acid sterol esters are placed in a vacuum oven operated at 2 mm Hg at a temperature of 55° C. for a period of 2 hours to remove any residual hexane, giving 9.2 grams of a clear viscous liquid. This material (the sterol ester composition) is analyzed by GC and is found to contain 97.8% sterol esters (94.4% unsaturated fatty acid sterol esters, 3.4% saturated fatty acid sterol esters) and 2.2% free sterol.

The solid on the sintered glass filter is washed once with 10° C. hexane and air dried. The resulting saturated fatty acid sterol esters are placed in a vacuum oven operated at 2 mm Hg at a temperature of 40° C. for a period of 2 hours to remove any residual hexane giving 0.64 grams of a waxy white solid.

### Example 6

### Fractional Distillation of Methyl Esters to Remove Saturates

10,000 lbs. (4,536 kg) methyl esters from Example 1 are added to the stillpot of a commercial scale, batch distillation still consisting of a 2500 gallon (9,462 L) stillpot, a 25 feet (7.6 m) tall, 12 feet (3.7 m) diameter packed column (structured packing) consisting of 10 stages along with an overhead condenser, a receiver vessel, and a vacuum system. The distillation is conducted at a pressure of 6 mm Hg at the top of the column, 13 mm Hg above the stillpot, and with a temperature of between 380° F. (193° C.) to 435° F. (224° C.) in the stillpot. A top cut of 1010 lbs. (458 kg) containing primarily the palmitic acid methyl esters is taken followed by 8160 lbs. (3,701 kg) of low saturate canola methyl esters leaving 833 lbs. (378 kg) as still bottoms for recycle. The product contains less than 2% palmitic methyl esters.

### Example 7

### Preparation of a Hypocholesterolemic Cooking or Salad Oil

Sterol ester composition from Example 3 (31.6 grams) and Crisco™ Brand canola oil (68.4 grams) are added to a 250 ml Erlenmeyer flask equipped with a nitrogen inlet and magnetic stir bar. The mixture is heated with stirring to 50° C. for 5 minutes under nitrogen purge. The mixture is cooled to room temperature under nitrogen purge and stoppered. The 13.8% sterol ester blend is placed in a 70° F. (21° C.) constant temperature room for one month. At the end of this period, no visible precipitate is observed.

### Example 8

### Preparation of a Salad Dressing

⅓ cup (80 ml) balsamic vinegar is placed in a blender along with 1 tablespoon (15 ml) sugar, 1 teaspoon (5 ml) dry mustard, ¼ teaspoon (1.25 ml) salt, and ⅛ teaspoon (0.6 ml) pepper. The mixture is processed for 30 seconds on low speed. ⅔ cup (160 ml) of the hypocholesterolemic salad and cooking oil from Example 7 is then added slowly to the mixture with the blender still running on low.

### Example 9

### Preparation of a Shortening

A preferred hypocholesterolemic shortening composition according to the invention is prepared by combining the hypocholestemic salad and cooking oil of Example 7 (78.3%) with a cottonseed or soybean or mixtures thereof intermediate melting fraction having an Iodine Value of 50 (12.4%), completely hardened soybean oil having an Iodine value of 4 (6.3%), and a soybean monoglyceride (3.0%). The shortening is plasticized by a freeze/pick process, and nitrogen gas is dispersed in the shortening for appearance.

### Example 10

### Preparation of a Peanut Spread

A peanut spread is prepared from the following total ingredient formulation:

| Ingredients | Wt. % |
|---|---|
| Peanuts | 83.90 |
| Sugar | 5.8 |
| Peanut Oil | 0.73 |
| Salt | 1.2 |
| Molasses | 0.5 |
| Stabilizer* | 1.85 |
| Emulsifier** | 0.3 |
| Sterol esters (winterized) | 5.72 |

*Hardened rapeseed oil blended with hydrogenated soybean oil
**Mono- and di-glycerides of palmitic and stearic acids

Peanut paste is made by comminuting roasted peanuts in a Bauer mill. To make a 300 pound (136 kg) batch of peanut butter, the following ingredients are mixed together in a 100 gallon (378 L) Hamilton kettle: 251.7 pounds (114 kg) of peanut paste, 2.19 pounds (0.99 kg) peanut oil, 1.5 pounds (0.68 kg) of molasses, 5.55 pounds (2.52 kg) of stabilizer, 3.60 pounds (1.63 kg) of salt, 17.4 pounds (7.89 kg) of sugar, and 17.16 pounds (7.98 kg) of sterol ester composition from Example 3. The ingredients are mixed for 30 minutes at 25 rpm and then passed through a Gaulin M-8 homogenizer at 4000 psig (276 Pa). The mixture is then processed through a deaerator (versator) and a scraped wall heat exchanger to increase the oxidative stability of the peanut butter and to set up its crystalline structure. The scraped wall heat exchanger is operated such that the freezer outer temperature is between 97° F. (36° C.) and 100° F. (38° C.). Chunks or pieces of nuts can also be added to the finished peanut spread at this point if desired. (See also U.S. Pat. Nos. 5,079,207; 5,508,057; 5,518,755; 5,667,838; 5,693,357; and 6,063,430, all to Wong et al.)

### Example 11

### Preparation of a Reduced Fat Peanut Spread

A monomodal reduced fat creamy peanut spread is prepared from a monomodal nut paste of the type described herein. The ingredients used to prepare this peanut spread are as follows:

| Ingredient | Wt. % |
|---|---|
| Peanuts | 61 |
| Molasses | 0.5 |
| Salt | 1.6 |
| Sugar | 6.4 |
| Stabilizer* | 1.25 |
| Emulsifier** | 0.75 |
| Soy Protein Isolate | 5 |
| Corn Syrup Solids | 18.3 |
| Vitamins/Minerals | 0.1 |
| Sterol ester composition from Example 3 | 5.1 |

*Hardened rapeseed oil blended with hydrogenated soybean oil
**Mono- and di-glycerides of palmitic and stearic acids

The peanuts are roasted at 422° F. (217° C.) and blanched and ground in a Bauer Mill. The ground peanuts are then pumped through a Rannie type #18.72H Homogenizer at a rate of 1200 lbs/hour (544 kg/hour) and at a pressure of 12,000 psig (827 Pa). The homogenized nut paste is then cooled by passing it through a heat exchanger and is deposited into a 100 gallon (378 L) Hamilton kettle.

The water insoluble particles comprising the nut paste have a monomodal particle size distribution such that 88% of the water insoluble solids comprising the nut paste have a particle size of less than about 21.6 microns, 80% of the water insoluble solids comprising the nut paste have a particle size of less than about 16.7 microns, 70% of the water insoluble solids comprising the nut paste have a particle size of less than about 13.0 microns, 59% of the water insoluble solids comprising the nut paste have a particle size of less than about 10.1 microns, 47% of the water insoluble solids comprising the nut paste have a particle size of less than about 7.9 microns, 31% of the water insoluble solids comprises the nut paste have a particle size of less than about 6.2 microns, 41% of the water insoluble solids comprising the nut paste have a particle size of about 10.1 microns or greater, 69% of the water insoluble solids comprising the nut paste have a particle size of about 6.2 microns or greater, and 90% of the water insoluble solids comprising the nut paste have a particle size of about 3.8 microns or greater. The particles size distribution curve of the non-water soluble solids comprising the nut paste is centered at 8.4 microns.

The molasses, stabilizer, and emulsifier and sterol ester composition are added to the mixing tank containing the nut paste, which is held at a constant temperature of 150° F. (66° C.). Mixing continues for about 5 minutes.

Salt and sugar are then loaded into a K-Tron-35 Twin Screw feeder positioned over the mixing tank and added to the mixing tank at a constant feed rate of 103 lbs/hour (46.7 kg/hour). After the sugar and salt have been added, corn syrup solids are loaded into the feeder and then added to the mix tank at the same rate. Lastly, the soy protein isolate is loaded into the feeder and added to the mix tank at the same rate.

Throughout the time that the solids are being added to the peanut paste in the mixing tank, a portion of the tank mixture is pumped through a 5 inch (12.7 cm) Greerco W-500 H Colloid Mill operated at a wide open gap, a heat exchanger and then redeposited in the mixing tank. This is a recirculating loop at 1200 lbs/hour (544 kg/hour). After all of the solids have been added, the mixture continues to be recycled through the colloid mill and heat exchanger for 30 minutes.

The mixture is then pumped through a Rannie type #18.72H homogenizer at a pressure of 12,000 psig (827 Pa) and then a heat exchanger and a colloid mill and deposited into a tank. The vitamins and minerals are added to the mixture, and the mixture is passed through a conventional peanut butter finishing system. For example, the mixture can be passed through a versator and a scraped wall heat exchanger, and then cooled and passed through picker boxes. Preferably, the temperature is below 50° C.

The finished nut spread has a Casson plastic viscosity of about 17.3 poise and a yield value of 198 dynes per square centimeter. The water insoluble solids comprising the peanut spread product have a monomodal particle size distribution such that 92% of the water insoluble solids comprising the nut spread have a particle size of less than about 21.6 microns, 88% of the water insoluble solids comprising the nut spread have a particle size of less than about 16.7 microns, 80% of the water insoluble solids comprising the nut spread have a particle size of less than about 13.0 microns, 66% of the water insoluble solids comprising the nut spread have a particle size of less than about 10.1 microns, 50% of the water insoluble solids comprising the nut paste have a particle size of less than about 7.9 microns, 32% of the water insoluble solids comprises the nut spread have a particle size of less than about 6.2 microns, 34% of the water insoluble solids comprising the nut paste have a particle size of about 10.1 microns or greater, 68% of the water insoluble solids comprising the nut paste have a particle size of about 6.2 microns or greater, and 89% of the water insoluble solids comprising the nut paste have a particle size of about 3.8 microns or greater. The particles size distribution curve of the non-water soluble solids comprising the nut spread is centered at 7.8 microns. The fat content of the nut spread is 34%. (See also U.S. Pat. Nos. 5,079,207; 5,508,057; 5,518,755; 5,667,838; 5,693,357; and 6,063,430, all to Wong et al.)

### Example 12

### Preparation of a Hypocholestemic Salad and Cooking Oil Made with Diglyceride Oil

Sterol ester composition from Example 3 (21.1 grams) and diglyceride oil (78.9 grams) are added to a 250 ml Erlenmeyer flask equipped with a nitrogen inlet and magnetic stir bar. The mixture is heated with stirring to 50° C. for 5 minutes under nitrogen purge. The mixture is cooled to room temperature under nitrogen purge and stoppered. The 9.2% sterol ester blend is placed in a 70° F. (21° C.) constant temperature room for one month. At the end of this period, no visible precipitate is observed.

### INCORPORATION BY REFERENCE

All of the aforementioned patents, publications, and other references are herein incorporated by reference in their entirety.

What is claimed is:

1. A sterol ester composition, wherein more than 50% of the fatty acid moieties are MUFAs.

2. The sterol ester composition of claim 1, wherein from about 55% to about 80% of the fatty acid moieties are MUFAs.

3. The sterol ester composition of claim 2, wherein from about 60% to about 70% of the fatty acid moieties are MUFAs.

4. The sterol ester composition of claim 1, wherein less than about 6% of the fatty acid moieties are SFAs.

5. The sterol ester composition of claim 4, wherein from about 0. 1% to about 4% of the fatty acid moieties are SFAs.

6. The sterol ester composition of claim 5, wherein from about 0.5% to about 2% of the fatty acid moieties are SFAs.

7. The sterol ester composition of claim 6, wherein 50% or less of the fatty acid moieties are PUFAs.

8. A capsule comprising the sterol ester composition of claim 1.

9. A capsule comprising the sterol ester composition of claim 2.

10. A capsule comprising the sterol ester composition of claim 4.

11. A capsule comprising the sterol ester composition of claim 6.

12. A capsule comprising the sterol ester composition of claim 7.

13. A food product comprising the sterol ester composition of claim 1.

14. The food product of claim 13, wherein said food product is a salad oil or cooking oil.

15. The food product of claim 14, wherein said salad oil or cooking oil comprises from about 5% to about 30% of said sterol ester composition.

16. The food product of claim 15, wherein said salad oil or cooking oil comprises from about 10% to about 20% of said sterol ester composition.

17. The food product of claim 13, wherein said food product is selected from the group consisting of shortenings, peanut butters, peanut spreads, mayonnaise, sauces, gravies, margarine, health bars, snacks, beverages, ice cream, yogurt, cake mix, frosting, donuts, baked goods, cheese, and cheese spreads.

18. A food product comprising the sterol ester composition of claim 2.

19. The food product of claim 18, wherein said food product is a salad oil or cooking oil.

20. The food product of claim 19, wherein said salad oil or cooking oil comprises from about 5% to about 30% of said sterol ester composition.

21. The food product of claim 20, wherein said salad oil or cooking oil comprises from about 10% to about 20% of said sterol ester composition.

22. The food product of claim 18, wherein said food product is selected from the group consisting of shortenings, peanut butters, peanut spreads, mayonnaise, sauces, gravies, margarine, health bars, snacks, beverages, ice cream, yogurt, cake mix, frosting, donuts, baked goods, cheese, and cheese spreads.

23. A food product comprising the sterol ester composition of claim 4.

24. The food product of claim 23, wherein said food product is a salad oil or cooking oil.

25. The food product of claim 24, wherein said salad oil or cooking oil comprises about 5% to about 30% of said sterol ester composition.

26. The food product of claim 25, wherein said salad oil or cooking oil comprises from about 10% to about 20% of said sterol ester composition.

27. The food product of claim 23, wherein said food product is selected from the group consisting of shortenings, peanut butters, peanut spreads, mayonnaise, sauces, gravies, margarine, health bars, snacks, beverages, ice cream, yogurt, cake mix, frosting, donuts, baked goods, cheese, and cheese spreads.

28. A food product comprising the sterol ester composition of claim 6.

29. The food product of claim 28, wherein said food product is a salad oil or cooking oil.

30. The food product of claim 29, wherein said salad oil or cooking oil comprises from about 5% to about 30% of said sterol ester composition.

31. The food product of claim 30, wherein said salad oil or cooking oil comprises from about 10% to about 20% of said sterol ester composition.

32. The food product of claim 28, wherein said food product is selected from the group consisting of shortenings, peanut butters, peanut spreads, mayonnaise, sauces, gravies, margarine, health bars, snacks, beverages, ice cream, yogurt, cake mix, frosting, donuts, baked goods, cheese, and cheese spreads.

33. A food product comprising the sterol ester composition of claim 7.

34. The food product of claim 33, wherein said food product is a salad oil or cooking oil.

35. The food product of claim 34, wherein said salad oil or cooking oil comprises from about 5% to about 30% of said sterol ester composition.

36. The food product of claim 35, wherein said salad oil or cooking oil comprises from about 10% to about 20% of said sterol ester composition.

37. The food product of claim 33, wherein said food product is selected from the group consisting of shortenings, peanut butters, peanut spreads, mayonnaise, sauces, gravies, margarine, health bars, snacks, beverages, ice cream, yogurt, cake mix, frosting, donuts, baked goods, cheese, and cheese spreads.

38. The sterol ester composition of claim 1, 2, 4, 6, or 7, wherein the free sterol level is less than about 10%.

39. The sterol ester composition of claim 38, wherein the free sterol level is less than about 5%.

40. The sterol ester composition of claim 39, wherein the free sterol level is less than about 3%.

41. A salad oil or cooking oil that is free of solids at temperatures of greater than about 60° F. and that comprises more than about 10% sterol esters.

42. A method for preparing the sterol ester composition of claim 1, comprising subjecting a sterol ester mixture to a fractionating process selected from the group consisting of solvent fractionation and fractional distillation of the fatty acid lower alkyl esters.

43. The method of claim 42, wherein the sterol ester mixture is produced by esterifying a fatty acid mixture with free sterol by a direct esterification method selected from the group consisting of catalytic esterification and enzymatic esterification.

44. The method of claim 42, wherein the sterol ester mixture is produced by esterifying an alcohol ester of a fatty acid mixture with free sterol in the presence of an interesterification catalyst.

45. A method for preparing the sterol ester composition of claim 1, comprising fractionating a mixture of sterol ester and vegetable oil.

46. A method for preparing the sterol ester composition of claim 1, comprising:
(a) esterifying a sterol with an excessive amount of a fatty acid alcohol ester in the presence of an interesterification catalyst; and
(b) fractionating the sterol fatty acid ester in the fatty acid alcohol ester to obtain the sterol ester composition.

47. The method of claim 42, comprising the additional steps of washing, drying, and bleaching the sterol fatty acid ester and fatty acid alcohol ester mixture before the fractionating process.

48. A method for preparing the sterol ester composition of claim 1, comprising:
(a) providing fatty acid esters;
(b) preparing a sterol ester mixture;
(c) refining the sterol ester mixture;
(d) evaporating and stripping the sterol ester mixture; and
(e) fractionating the sterol ester mixture to obtain the sterol ester composition.

49. A method for preparing the sterol ester composition of claim 1, comprising:
(a) providing fatty acid esters;
(b) preparing a sterol ester mixture;
(c) refining the sterol ester mixture;
(d) evaporating and stripping the sterol ester mixture;
(e) adding a nucleating agent; and
(e) fractionating the sterol ester mixture to obtain the sterol ester composition.

50. A method for preparing a salad oil or cooking oil comprising the sterol ester composition of claim 1, comprising:
(a) interesterifying a sterol and an edible oil with an excess amount of fatty acid alcohol ester in the presence of an interesterification catalyst to form a sterol fatty acid ester mixture; and
(b) fractionating the sterol fatty acid ester mixture to obtain the salad oil or cooking oil.

51. The capsule of claim 8, 9, 10, 11, or 12, wherein the sterol ester composition has a free sterol level of less than about 10%.

52. The capsule of claim 51, wherein the sterol ester composition has a free sterol level of less than about 5%.

53. The capsule of claim 52, wherein the sterol ester composition has a free sterol level of less than about 3%.

54. The food product of claim 13, 18, 23, 28, or 33, wherein the sterol ester composition has a free sterol level of less than about 10%.

55. The food product of claim 54, wherein the sterol ester composition has a free sterol level of less than about 5%.

56. The food product of claim 55, wherein the sterol ester composition has a free sterol level of less than about 3%.
